(19) United States
(12) Patent Application Publication
(10) Pub. No.: US 2009/0221978 A1
(43) Pub. Date: Sep. 3, 2009

(54) ABSORBENT ARTICLE WITH LOTION COMPRISING A POLYPROPYLENE GLYCOL MATERIAL

(76) Inventors: Joseph Anthony Gatto, Redondo Beach, CA (US); Brent Taylor Ginn, Monroe, OH (US); Robert Ya-lin Pan, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/369,922

(22) Filed: Feb. 12, 2009

Related U.S. Application Data
(60) Provisional application No. 61/029,165, filed on Feb. 15, 2008.

(51) Int. Cl. A61F 13/15

(52) U.S. Cl. 604/367; 604/385.23; 604/385.01

(57) ABSTRACT

An absorbent article, such as a catamenial device, comprises a liquid pervious topsheet, the topsheet having an inner surface oriented toward the interior of the absorbent article and an outer surface oriented toward the skin of the wearer when the absorbent article is being worn. The absorbent article includes a backsheet joined to the topsheet, the backsheet having an inner surface oriented toward the interior of the absorbent article and an outer surface oriented toward the garment of the wearer when the absorbent article is being worn. The absorbent article includes an absorbent core disposed between the topsheet and the backsheet, the absorbent core having an inner surface oriented toward the skin of the wearer when the absorbent article is being worn and an outer surface oriented toward the garment of the wearer when the absorbent article is being worn. The absorbent article includes a lotion composition applied to at least a portion of the outer surface of the topsheet, wherein the lotion composition comprises a polypropylene glycol material.

### CROSS REFERENCE TO RELATED APPLICATIONS

This application claims the benefit of U.S. Provisional Application No. 61/029,165, filed Feb. 15, 2008.

### FIELD OF INVENTION

This application relates to absorbent articles, including catamenial devices such as tampons and sanitary napkins for the absorption of menses. More particularly, the present invention relates to catamenial devices comprising a lotion composition comprising a polypropylene glycol material, the lotion composition being applied to an outer surface thereof.

### BACKGROUND OF THE INVENTION

Disposable absorbent articles, such as diapers, training pants, and catamenial devices having lotioned topsheets are known. Lotions of various types are known to provide various skin benefits, such as prevention or treatment of diaper rash. These lotions can be applied to the topsheet of absorbent articles, for example, and can be transferred to the skin of the wearer during use.

Unlike many types of disposable absorbent articles, catamenial devices such as pads and pantiliners are specifically designed to acquire menstrual fluid. Menstrual fluid differs from other exudates, such as urine, in many important properties, such as viscosity and solids, chemical, and/or color content. Therefore, catamenial devices should differ in their structural components from such devices as baby diapers to be optimized for the maximum absorption of menstrual fluid.

Treatments to the bodyside surface of the topsheet of diaper absorbent products have been proposed to primarily provide skin health benefits and secondarily to allow fluid such as urine to be absorbed into the product. Skin health benefits may include reduced direct contamination of body fluids in contact with the body surface and/or delivery of ingredients to improve skin function, i.e., skin barrier. Nonetheless these treatments are known to impede the acquisition of bodily fluids and ‘schemes’ have been designed to place these treatments on regions of the bodyside surface of the topsheet to minimize their effect on fluid acquisition. Treatments of the bodyside surface of the topsheet of feminine hygiene products have also been proposed to provide skin health benefits and similarly the treatment deployment is generally performed to minimize the hindrance of menstrual fluid acquisition. Nonetheless, the use of these topsheet treatments often becomes ineffective after repeated assaults of bodily fluid due to deposition of blood cells or other soils on the topsheet surface. In addition, the treatment sometimes can provoke negative product failure signals to the user of feminine hygiene products including the appearance of a dirty or soiled topsheets or negative odor sensorial signals. This type of treatment on a topsheet may provide a compromised skin health benefit and a non-acceptable appearance of a used feminine hygiene product.

It would be beneficial to have a treatment for feminine hygiene articles that can enable migration of fluid away from the body and through the topsheet into the product.

Additionally, it would be beneficial to have a treatment for feminine hygiene articles that enables migration of fluid, such as menstrual fluid, in a controlled fashion.

Further, it would be beneficial to have a treatment for feminine hygiene articles that facilitates fluid, such as menstrual fluid, being moved so as to enhance perceived cleanliness of both the topsheet and of the skin of the wearer, especially after multiple assaults of bodily fluid.

### SUMMARY OF THE INVENTION

An absorbent article, such as a catamenial device, comprises a liquid pervious topsheet, the topsheet having an inner surface oriented toward the interior of the absorbent article and an outer surface oriented toward the skin of the wearer when the absorbent article is being worn. The absorbent article includes a backsheet joined to the topsheet, the backsheet having an inner surface oriented toward the interior of the absorbent article and an outer surface oriented toward the garment of the wearer when the absorbent article is being worn. The absorbent article includes an absorbent core disposed between the topsheet and the backsheet, the absorbent core having an inner surface oriented toward the skin of the wearer when the absorbent article is being worn and an outer surface oriented toward the garment of the wearer when the absorbent article is being worn. The absorbent article comprises a lotion composition applied to at least a portion of the outer surface of the topsheet, with the lotion composition comprising a polypropylene glycol material. In one embodiment, the lotion composition consists essentially of a polypropylene glycol material, especially a polypropylene glycol homopolymer such as polypropylene glycol, and optionally a carrier. In another embodiment, the lotion composition comprises a polypropylene glycol material selected from the group consisting of polypropylene glycol copolymer, polypropylene glycol surfactant, and mixtures thereof. The lotion composition comprising the polypropylene glycol material as applied to the outer surface of the topsheet helps to resist the adherence of bodily fluid, such as menses, to the topsheet and upon transfer to the wearer of the absorbent article, resist the adherence of bodily fluid to the skin of the wearer, thereby resulting in less staining on the topsheet of the absorbent article and less fouling of the skin of the wearer. The lotion composition as applied to the outer surface of the topsheet can also help to improve continuous fluid acquisition of the absorbent article.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a top view of a representative catamenial device having a topsheet, backsheet, and an absorbent core.

FIG. 2 is a top view of another representative catamenial device having a topsheet, a backsheet, and an absorbent core.

FIG. 3 is a top view of another representative catamenial device having a topsheet, a backsheet, and an absorbent core.

### DETAILED DESCRIPTION OF THE INVENTION

The present invention is directed to absorbent articles, particularly disposable absorbent articles, having thereon a lotion treatment composition, the treatment composition comprising a polypropylene glycol (“PPG”) material. Disposable absorbent articles can be baby diapers or feminine hygiene articles, including incontinence devices and catamenial products such as tampons, sanitary napkins, pantiliners, interlabial products, and the like. The invention is disclosed below with respect to one embodiment of a catamenial device, such as a sanitary napkin or pantiliner.

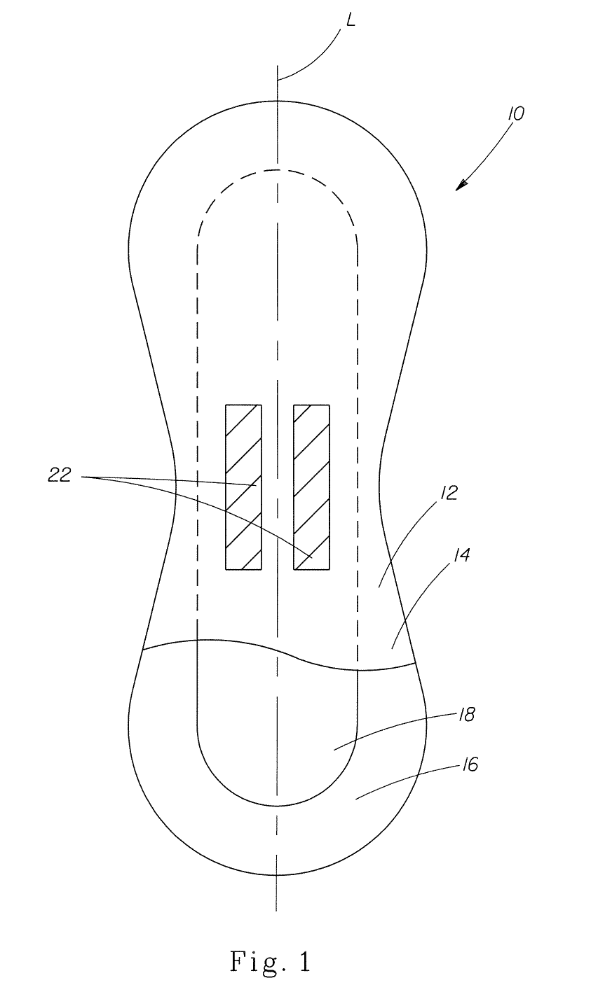

FIGS. 1, 2, and 3 show a catamenial device 10, that can be a sanitary napkin or pantiliner, having a body-contacting surface 12 comprising the outer surface of a topsheet 14, a liquid impervious backsheet 16 joined to the topsheet 14, an absorbent core 18. The sanitary napkin 10 has a longitudinal axis L and may also be provided with additional features commonly found in napkins, including “wings” or “flaps” (not shown) as is known in the art, and, and/or a secondary topsheet, and/or a fluid acquisition layer, and/or other layers designed to promote fluid transport to the absorbent core 18. Likewise, the topsheet of the sanitary napkin can have various optional characteristics, as is known in the art. For example, the topsheet 14 can have channels embossed therein to direct fluid flow, and can have apertures there through to aid in fluid acquisition, and can have printed signals visible on or through, the visible signals being printed on the topsheet or underlying layers, and visible for functional and aesthetic properties.

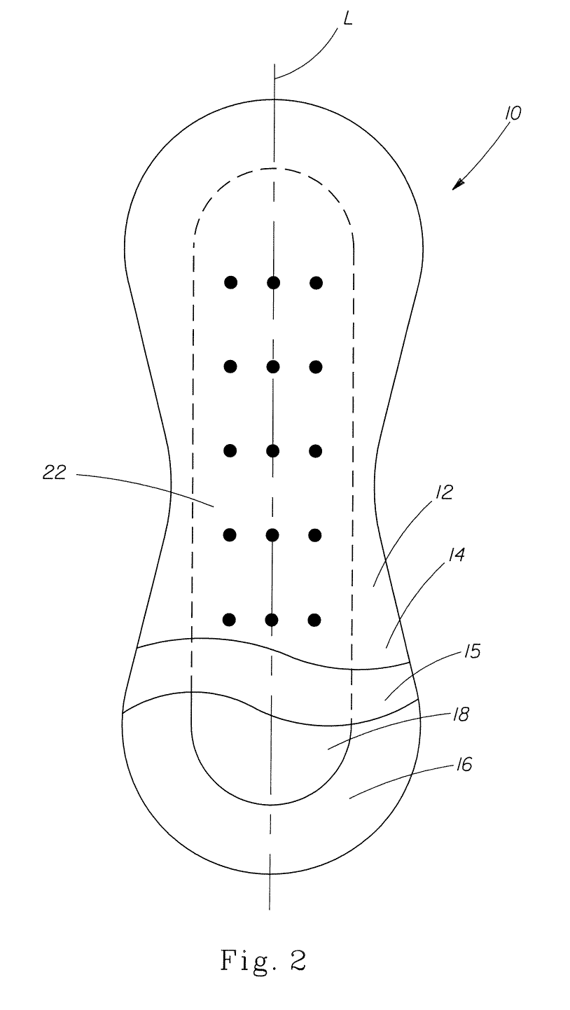

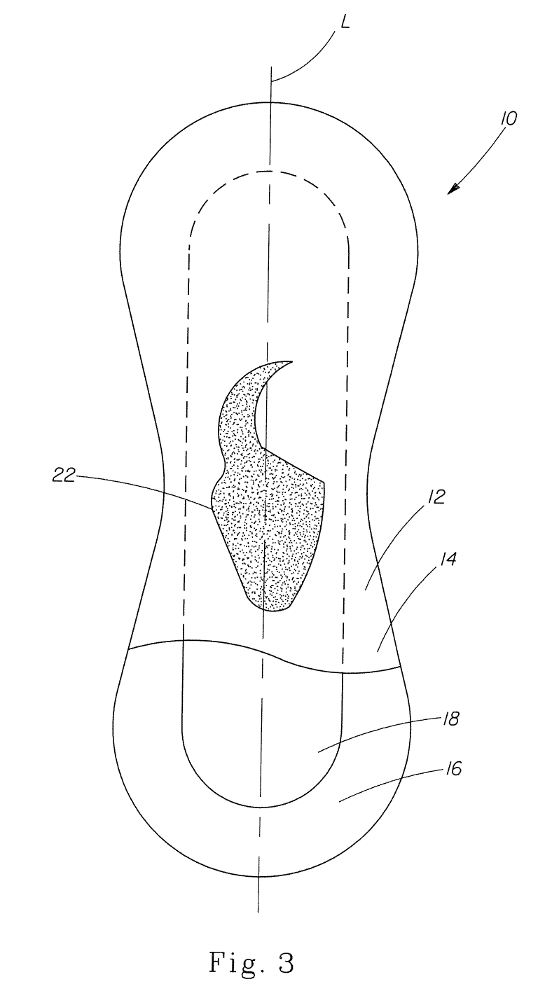

The catamenial devices 10 of FIGS. 1, 2, and 3 each have a lotion composition 22 applied thereto. The catamential device 10 of FIG. 1 has a lotion composition 22 applied in parallel stripes to the outer surface of the topsheet 14. The catamenial device 10 of FIG. 2 has a lotion composition 22 applied in a pattern of dots to the outer surface of the topsheet 14. The catamenial device 10 of FIG. 3 has a lotion composition 22 applied in a non-uniform pattern to the outer surface of the topsheet 14.

The absorbent article may comprise any known or otherwise effective topsheet, such as one which is compliant, soft feeling, and non-irritating to the wearer's body. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, also provides for the transfer or migration of the lotion composition onto an external or internal portion of a wearer's body. A suitable topsheet can be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making catamenial products such as sanitary napkins, pantiliners, incontinence pads, and the like.

When the topsheet comprises a nonwoven fibrous material in the form of a nonwoven web, the nonwoven web may be produced by any known procedure for making nonwoven webs, nonlimiting examples of which include spunbonding, carding, wet-laid, air-laid, meltblown, needle-punching, mechanical entangling, thermo-mechanical entangling, and hydroentangling.

The catamenial device of the present invention also comprises a backsheet. The backsheet can be any known or otherwise effective backsheet material, provided that the backsheet prevents external leakage of exudates absorbed and contained in the catamenial device. Flexible materials suitable for use as the backsheet include, but are not limited to, woven and nonwoven materials, laminated tissue, polymeric films such as thermoplastic films of polyethylene and/or polypropylene, composite materials such as a film-coated nonwoven material, or combinations thereof, as is well known in the art of making catamenial products such as sanitary napkins, pantiliners, incontinence pads, and the like.

The catamenial device also comprises an absorbent core. The absorbent core is typically positioned between the topsheet and the backsheet. As used herein, the term “absorbent core” refers to a material or combination of materials suitable for absorbing, distributing, and storing aqueous fluids such as urine, blood, menses, and water found in body exudates. The size and shape of the absorbent core can be altered to meet absorbent capacity requirements, and to provide comfort to the wearer/user. The absorbent core suitable for use in the present invention can be any liquid-absorbent material known in the art for use in absorbent articles, provided that the liquid-absorbent material can be configured or constructed to meet absorbent capacity requirements. Nonlimiting examples of liquid-absorbent materials suitable for use as the absorbent core include comminuted wood pulp which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making catamenial products such as sanitary napkins, pantiliners, incontinence pads, and the like.

The catamenial device of the present invention comprises a lotion composition 22, wherein the lotion composition comprises a PPG material. The PPG lotion composition 22 can be applied in any known manner, in any known pattern, and to the outer surface (i.e. body facing surface) of the topsheet 14. For example, the lotion 22 can be applied in a pattern of generally parallel stripes or bands, as depicted in FIG. 1. Outer surfaces can include surfaces such as the body-facing side of a sanitary napkin topsheet, or the vaginal wall facing surface of a tampon overwrap.

With respect to applying the lotion composition to the outer surface (i.e. body facing surface) of the topsheet, it is believed that the PPG lotion composition helps to reduce adherence of bodily fluid, such as menses, to the outer surface of the topsheet, thereby resulting in less staining on the topsheet of the absorbent article. Applying the PPG lotion to the topsheet also enables transfer of the PPG lotion to the skin of the wearer, resulting in less fouling of the skin of the wearer. The lotion composition as applied to the outer surface of the topsheet can also help to provide more efficient acquisition of the bodily fluid into the absorbent article.

For catamenial devices the amount of lotion add on level can be variable, and can be tailored for specific needs. For example, while not being bound by theory, it is believed that lotion can be added on at levels of about 0.01 grams per square meter (“gsm”), about 0.05 gsm, about 0.1 gsm, about 0.5, about 1 gsm, about 2 gsm, about 3 gsm, about 4 gsm, about 5 gsm, about 10 gsm, about 25 gsm, about 50 gsm, or about 100 gsm. The lotion can be applied within a range defined by any of the levels recited above (e.g. from about 0.01 gsm to about 100 gsm). These levels refer to the area of the surface actually covered by lotion.

The catamenial device and lotion composition device include components, structure, patterns, formulations, and variations as described in US 2005/0129651 A1, US 2004/0170589 A1, and US 2005/0208113 A1.

The lotion composition 22 of the present invention offers significant advantages over known lotions, including non-PPG derived surfactants and other surface modifying agents. The advantage is particularly important for catamenial articles for absorbing menses. Without being bound by theory, it is believed that the superior fluid handling properties of the PPG materials herein is due to the way the PPG materials act on the solid components of menses as opposed to surface energy treatments which act on the water component of menses. Known surface energy treatments are less effective due to the presence of polar and dispersive components in menses, which complicates the effectiveness of surface energy treatments. Because the PPG materials herein are typically not readily soluble in menses, it can effectively coat surfaces without dissolving in menses, which provides a hydrated barrier whose electron donating dipoles repulse negatively dipoled proteins, thereby rendering the menses less prone to fouling surfaces of the disposable absorbent article or the wearer's skin. Less fouling of the wearer's skin and/or topsheet results in better and faster fluid movement, and less visible stain patterns on used products.

The PPG materials herein can be applied as one component in a lotion composition, or can be applied neat (i.e. the lotion composition consists of PPG material). PPG materials, either neat and/or as part of a lotion composition, can be applied at varying add-on levels, depending on the fluid handling properties desired and desired treatment of the wearer's skin. PPG materials are added to the outer surface of the topsheet in any pattern, such as full coat, stripes or bands (in the MD or CD direction), droplets, spiral patterns, and the like. The lotion composition comprising the PPG material can also be disposed near channels or embossed areas when present in the absorbent article.

In one embodiment, the topsheet 14 can be hydrophilic or rendered to be hydrophilic, and the lotion can be also hydrophilic. The levels of hydrophilicity or hydrophobicity can be determined by standard techniques, such as measuring angles that a drop of water make on a surface of material at equilibrium. In general, for the purposes of this invention, a material is considered hydrophilic if a drop of water exhibits an angle of about 60 degrees or less. Fibers are considered to be hydrophilic if film sheets formed from the polymers of the fibers would exhibit contact angles with water less than 60 degrees, more less than 75 degrees, and less than about 90 degrees. Contact angles as a measure of hydrophilicity are well known in the art, and methods for measuring contact angles are equally well known. As is well known, contact angles greater than about 90 degrees are considered hydrophobic.

The lotion composition of the present invention comprises a PPG material. The PPG materials suitable herein include PPG homopolymer materials, PPG copolymer materials, and PPG surfactant materials, as well as mixtures thereof. The lotion composition can further comprise other optional ingredients. In one embodiment, the lotion composition consists essentially of, or consists of, a PPG material, preferably polypropylene glycol. In another embodiment, the lotion composition comprises a PPG material selected from the group consisting of polypropylene glycol copolymer, polypropylene glycol surfactant, and mixtures thereof.

The lotion compositions of the present invention can comprise a PPG material at a level of from about 0.1% to about 100%, by weight of the lotion composition. In one embodiment, the lotion composition comprises less than about 10%, preferably from about 0.5% to about 8%, and more preferably from about 1% to about 6%, of a PPG material, by weight of the lotion composition. In another embodiment, the lotion composition comprises at least about 50%, preferably from about 75% to about 100%, and more preferably from about 90% to about 100%, of a PPG material, by weight of the lotion composition.

Suitable PPG homopolymer materials include those corresponding to the following formula:

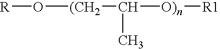

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R is preferably hydrogen or methyl;
wherein R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R1 is preferably hydrogen or methyl; and
wherein n is from 3 to 160, preferably from 5 to 120, more preferably from 10 to 100, and more preferably from 20 to 80.

Optionally, the PPG homopolymer may include low level of glycerol or butanediol as part of its monomer raw material. If in presence, the preferred ratio of glycerol or butanediol to propylene glycol ranges from 1:1000 to 1:2, most preferably 1:100 to 1:5. The PPG homopolymer can have, but not limited to, the CAS Number 25322-69-4 and 25791-96-2.

Non-limiting examples of suitable PPG homopolymer materials are polypropylene glycol 4000 such as Pluriol P-4000 (BASF), Alkapol PPG-4000 (Alkaril Chemical) and Niax Polyol PPG 4025 (Union Carbide); polypropylene glycol 3500; polypropylene glycol 3000 such as Niax PPG 3025 (Union Carbide); polypropylene glycol 2000 such as Alkanol PPG-2000 (Alkaril Chemical) and Pluriol P-2000 (BASF), polypropylene glycol 1200 such as Alkapol PPG-1200 (Alkaril Chemical) and Glucam P-20 Humectant (Noveon); polypropylene glycol 1000 such as Niax PPG 1025 (Union Carbide); polypropylene glycol 600 such as Alkanol PPG-600 (Alkaril Chemical) and Glucam P-10 Humectant (Noveon); polypropylene glycol 400 such as Alkanol PPG-425 (Alkaril Chemical). polypropylene glycol 4000 glycerol ether such as Pluriol T-4000 (BASF); polypropylene glycol 2000 glycerol ether, polypropylene glycol 2000 butanediol ether, polypropylene glycol 1500 glycerol ether such as Pluriol T-1500 (BASF), polypolypropylene glycol 4000 with monomethyl ether, polypropylene glycol 2000 with dimethyl ether, polypropylene glycol 4000 with monobutyl ether, polypropylene glycol 2000 with monobuytyl ether, polypropylene glycol 1200 with dibutyl ether, polypropylene glycol 4000 with bis(2-aminopropyl ether), PPG-10 methyl glucose ether and PPG-20 methyl glucose either.

Suitable PPG homopolymer materials will typically have a number average molecular weight of from about 400 to about 10,000, preferably from about 600 to about 6,000, and more preferably from about 1,200 to about 4,800.

Suitable PPG copolymer materials include those in which the polyprolyene glycol segments are present as an internal block component and/or as a terminal component, of the copolymer structure. The following formulae illustrate the internal block components and terminal block components:

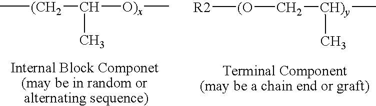

wherein x is from 2 to 120, preferably from 2 to 80, and more preferably from 3 to 60; y is from 2 to 100, preferably from 2 to 50, and more preferably from 3 to 30; R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate, and wherein R2 is preferably hydrogen, methyl, ethyl, isopropyl or isobutyl.

The polymers suitable to form propoxylated copolymers with PPG for the present lotion compositions include homopolymers of alkyl methicone, phenyl methicone, dimethicone, alkyl trimethicone, phenyl trimethicone, polyol, polyether (e.g. polyoxymethylene, polyoxyethylene and polyoxypropylene), polyimine, polyamide, polyacrylate, polyester, and copolymers containing one or multiple of these polymeric units. Non-limiting examples of suitable polymers include polydimethyl siloxane, polyethylimine, polyacrylic acid, poly(ethylene-co-acrylic acid), polymethacrylic acid, poly(ethylene-co-methacrylic acid), poly(vinyl acetate), polyvinylpyrrolidone, poly(ethylene-co-vinyl acetate), poly(butanediol), poly(neopentyl glycol), poly(ethylene adipate), poly(butylene adipate), poly(ethylene glutamate), poly(butylene glutamate), poly(ethylene sebacate), poly(butylene sebacate), poly(ethylene succinate), poly(butylene succinate), poly(ethylene terephthalate), poly(butylene terephthalate), polycaprolactone, and polyglycerol.

Non-limiting examples of suitable PPG copolymer materials include PPG-12 dimethicone such as Sisoft 910 (Momentive); bis-PPG-15 dimethicon/IPDI copolymer such as Polyderm-PPI-SI-WI (Alzo); PPG/polycaprolactone block copolymer; PPG/polybutanediol/PEG triblock copolymer; polyethylimine/PPG copolymer and polyacrylic acid-g-PPG graft copolymer.

Another suitable PPG material includes PPG surfactant materials. The following formula represents suitable PPG surfactant materials wherein the PPG segments constitute a part of the head functional group:

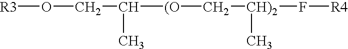

wherein R3 is hydrogen, alkyl, alkyl carbonyl, alkylenelamine, alkylenelamide, alkylene phosphate, alkylene carboxylic acid, alkylene sulfonate salt and alkylene quat with the maximum number of carbon element less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100, preferably from 2 to 30, and more preferably from 3 to 25; and F is a functional group selected from the group consisting of ether groups (including oxy, glyceryl, glucose, sorbitan, sucrose, monoethanolamine or diethanolamine), ester groups (including ester, glyceryl ester, glucose ester, sorbitan ester or sucrose ester), amine groups, amide groups, and phosphate ester groups.

The following formula represents suitable PPG surfactant materials wherein the PPG segments constitute an internal block group:

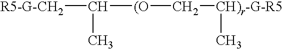

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120, preferably from 4 to 50, and more preferably from 6 to 30; and G is ether, ester, amine, or amide linkage.

Non-limiting examples of suitable PPG surfactant materials include PPG-30 cetyl ether such as Hetoxol C30P (Global Seven); PPG-20 methyl glucose ether distearate such as Glucam P-20 Distearate Emollient (Noveon), PPG-20 methyl glucose ether acetate, PPG-20 sorbitan tristearate, PPG-20 methyl glucose ether distearate, PPG-20 distearate, PPG-15 stearyl ether such as Alamol-E (Croda-Uniqema) and Procetyl 15 (Croda), PPG-15 stearyl ether benzoate, PPG-15 isohexadecyl ether, PPG-15 stearate, PPG-15 dicocoate, PPG-12 dilaurate, PPG-11 stearyl ether such as Varonic APS (Evonik); PPG-10 cetyl ether such as Procetyl 10 (Croda); PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether such as Promyristyl PM-3 (Croda), PPG-3 myristyl ether propionate such as Crodamol PMP (Croda), PPG-3 benzyl ether myristate such as Crodamol STS (Croda), PPG-3 hydrogenated castor oil such as Hetester HCP (Alzo), PPG-3-hydroxyethyl soyamide, PPG-2 Cocamide, PPG-2 lanolin alcohol ether and PPG-1 coconut fatty acid isopropanolamide such as Amizett IPC (Kawaken Fine Chemicals).

In addition to PPG materials, the lotion compositions of the present invention can optionally further comprise a select combination of body treatment agents such as hexamidine, zinc oxide, and niacinamide which are highly effective in the prevention and treatment of erythema, malodor, and bacterial skin disorders, especially when these lotion compositions are administered to the body from application on absorbent articles.

The lotion compositions of the present invention can optionally further comprise a carrier for the PPG material and/or optional body treatment agents. The carrier can be included in the compositions as an individual carrier or a combination of carrier ingredients, provided that the total carrier concentration is sufficient to provide transfer and/or migration of the PPG material and/or optional body treatment agents onto the body and to promote fluid acquisition into the absorbent article. The carrier can be a liquid, solid, or semisolid carrier material, or a combination of these materials, provided that the resultant carrier forms a homogenous mixture, solution, stable emulsion, or stable dispersion at selected processing temperatures for the resultant carrier system and at processing temperatures for combining the carrier with the skin treatment agents in formulating the lotion compositions herein. Processing temperatures for the carrier system may range from about 60° C. to about 90° C., more typically from about 70° C. to about 85° C., even more typically from about 70° C. to about 80° C.

The lotion compositions of the present invention can comprise the carrier at a total carrier concentration ranging from about 60% to about 99.9%, preferably from about 70% to about 98%, more preferably from about 80% to about 97% by weight of the lotion composition. Suitable carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, and mixtures thereof. Alternatively or in combination with, the carrier may also be composed of polysiloxane compounds non-limiting examples include dimethicones (1-100,000,000 centistoke), cyclomethicones, alkylated silicones (hair conditioning agents), silicone gums, silicone gels, silicone waxes, copolymers of silicone (vinyl dimethicone polymers, phenyl vinyl dimethicone polymers, alkylated silicone polymers, polyethylene oxide/silicone copolymers, polyethylene oxide/alkyl silicone copolymers), and mixtures thereof.

Nonlimiting examples of suitable petroleum-based hydrocarbons having from about 4 to about 32 carbon atoms include mineral oil, petrolatum, isoparaffins, various other branched chained hydrocarbons, and combinations thereof. Mineral oil is also known as “liquid petrolatum”, and usually refers to less viscous mixtures of hydrocarbons having from about 16 to about 20 carbon atoms. Petrolatum is also known as “mineral wax”, “petroleum jelly”, and “mineral jelly”, and usually refers to more viscous mixtures of hydrocarbons having from about 16 to about 32 carbon atoms. An example of commercially available petrolatum include petrolatum sold as Protopet® 1S which is available from the Witco Corporation located in Greenwich, Conn.

Other carriers suitable herein can include oils or fats such as natural oils or fats, or natural oil or fat derivatives, in particular of plant or animal origin. Non-limiting examples include avocado oil, apricot oil, apricot kernel oil, babassu oil, borage oil, borage seed oil, calendula oil, camellia oil, canola oil, carrot oil, cashew nut oil, castor oil, chamomile oil, cherry pit oil, chia oil, coconut oil, cod liver oil, corn oil, corn germ oil, cottonseed oil, eucalyptus oil, evening primrose oil, grape seed oil, hazelnut oil, jojoba oil, juniper oil, kernel oil, linseed oil, macadamia oil, meadowfoam seed oil, menhaden oil, mink oil, moring a oil, mortierella oil, olive oil, palm oil, palm kernel oil, peanut oil, peach kernel oil, rapeseed oil, rose hip oil, safflower oil, sandlewood oil, sesame oil, soybean oil, sunflower oil, sunflower seed oil, sweet almond oil, tall oil, tea tree oil, turnip seed oil, walnut oil, wheat germ oil, zadoary oil, or the hardened derivatives thereof. Hardened oils or fats from vegetal origin can include, e.g. hardened castor oil, peanut oil, soya oil, turnip seed oil, cottonseed oil, sunflower oil, palm oil, kernel oil, linseed oil, corn oil, olive oil, sesame oil, cocoa butter, shea butter and coconut oil.

Other non-limiting examples of fats and oils include: butter, C12-C18 acid triglyceride, camellia oil, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, human placental lipids, hybrid safflower oil, hybrid sunflower seed oil, hydrogenated castor oil, hydrogenated castor oil laurate, hydrogenated coconut oil, hydrogenated cottonseed oil, hydrogenated C12-C18 triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lanolin and lanolin derivatives, lanolin alcohol, lard, lauric/palmitic/oleic triglyceride, lesquerella oil, maleated soybean oil, meadowfoam oil, neatsfoot oil, oleic/linoleic triglyceride, oleic/palmitic/lauric/myristic/linoleic triglyceride, oleostearine, olive husk oil, omental lipids, orange roughy oil, pengawar djambi oil, pentadesma butter, phospholipids, pistachio nut oil, placental lipids, rapeseed oil, rice bran oil, shark liver oil, shea butter, sphingolipids, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, wheat bran lipids, and the like, as well as mixtures thereof.

Other suitable carriers include mono- or di-glycerides, such as those derived from saturated or unsaturated, linear or branch chained, substituted or unsubstituted fatty acids or fatty acid mixtures. Examples of mono- or diglycerides include mono- or di-C₁₂₋₂₄fatty acid glycerides, specifically mono- or di-C₁₆₋₂₀fatty acid glycerides, for example glyceryl monostearate, glyceryl distearate.

Carriers can also include esters of linear C₆-C₂₂-fatty acids with branched alcohols.

The carrier of the present invention can also include sterols, phytosterols, and sterol derivatives. Sterols and sterol derivatives that can be used in the lotion compositions of the invention include, but are not limited to: β-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

Nonlimiting examples of suitable fatty alcohols having from about 12 to about 24 carbon atoms include saturated, unsubstituted, monohydric alcohols or combinations thereof, which have a melting point less than about 110° C., preferably from about 45° C. to about 110° C. Specific examples of fatty alcohol carriers for use in the lotion compositions of the present invention include, but are not limited to, cetyl alcohol, stearyl alcohol, cetearyl alcohol, behenyl alcohol, arachidyl alcohol, lignocaryl alcohol, and combinations thereof. Examples of commercially available cetearyl alcohol is Stenol 1822 and behenyl alcohol is Lanette 22, both of which are available from the Cognis Corporation located in Cincinnati, Ohio.

Nonlimiting examples of suitable fatty acid esters include those fatty acid esters derived from a mixture of C₁₂-C₂₈ fatty acids and short chain (C₁-C₈, preferably C₁-C₃) monohydric alcohols preferably from a mixture of C₁₆-C₂₄ saturated fatty acids and short chain (C₁-C₈, preferably C₁-C₃) monohydric alcohols. Suitable fatty acid esters can also be derived from esters of longer chain fatty alcohols (C₁₂-C₂₈, preferably C₁₂-C₁₆) and shorter chain fatty acids such as lactic acid, specific examples of which include lauryl lactate and cetyl lactate. Representative examples of suitable fatty acid esters include methyl palmitate, methyl stearate, isopropyl laurate, isopropyl myristate, isopropyl palmitate, ethylhexyl palmitate, stearyl stearate, palmityl stearate, stearyl behenate, cetyl stearate, cetyl behenate, cetyl palmitate, cetearyl behenate, behenyl behenate, stearyl heptanoate, stearyl octanoate, myristyl myristate, myristyl isostearate, myristyl oleate, cetyl isostearate, cetyl oleate, stearyl isostearate, stearyl oleate, isostearyl myristat, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl isostearate, behenyl oleate, erucyl isostearate, and mixtures thereof.

Nonlimiting examples of suitable alkyl ethoxylates include C₁₂-C₂₂ fatty alcohol ethoxylates having an average degree of ethoxylation of from about 2 to about 30. Nonlimiting examples of suitable lower alcohols having from about 1 to about 6 carbon atoms include ethanol, isopropanol, butanediol, 1,2,4-butanetriol, 1,2 hexanediol, ether propanol, and mixtures thereof. Nonlimiting examples of suitable low molecular weight glycols and polyols include ethylene glycol, polyethylene glycol (e.g., Molecular Weight 200-600 g/mole), butylene glycol, propylene glycol, polypropylene glycol and mixtures thereof. A more detailed description of carrier ingredients including suitable hydrocarbons, polysiloxane compounds, and fatty alcohol ethoxylates can be found in U.S. Pat. No. 5,643,588, issued Jul. 1, 1997 to Roe et al. entitled “Diaper Having A Lotioned Topsheet”.

Suitable carriers further encompass waxes. As used herein, the term ‘wax’ refers to oil soluble materials that have a waxy constituency and have a melting point or range of above ambient temperature, in particular above 25° C. Waxes are materials that have a solid to semi-solid (creamy) consistency, crystalline or not, being of relative low viscosity a little above their liquefying point. Suitable waxes which can be incorporated into the lotion composition include animal, vegetable, mineral or silicone based waxes which may be natural or synthetic, and including mixtures thereof. Waxes can include but are not limited to: natural waxes from vegetal origin, such as bayberry wax, beeswax, candelilla wax, carnauba, ceresin, purcelline, shea butter, cocoa butter, Japan wax, jojoba wax, lanolin wax, esparto gras wax, cork wax, guaruma wax, rice shoot wax, ouricury wax, mink wax, montan wax, rice bran wax, spent grain wax, spermaceti wax, sunflower wax, ceresine wax, sugar cane wax, carnauba wax, candelilla wax, fruit-derived waxes, such as orange wax, lemon wax, grapefruit wax and bayberry wax, and the like; and waxes from animal origin such as beeswax, woolwax, bear fat, shellac wax, and the like. Natural waxes further comprise mineral waxes such as ceresin and ozokerite waxes. Synthetic waxes comprise petroleum-based waxes, such as certain carrier materials described hereinbefore, such as paraffin, vaseline, petrolatum, micro wax, and microcrystalline wax. Further suitable synthetic waxes are polyalkylene and polyethyleneglycol waxes, e.g. polyethylene wax; waxes based on chlorinated naphtalenes such as ‘Halowax’, synthetic hydrocarbon waxes, and the like, PEG-6 beeswax, PEG-8 beeswax, C30 alkyl dimethicone, synthetic beeswax, synthetic candelilla wax, synthetic carnuba wax, synthetic japan wax, synthetic jojoba wax, motan acid wax, motan wax, ouricury wax, rezowax, including mixtures thereof. Further suitable waxes are chemically modified waxes, in particular hardened or hydrogenated waxes such as, for example, hydrogenated cottonseed oil, hydrogenated jojoba oil, hydrogenated jojoba wax, hydrogenated microcrystalline wax, hydrogenated rice bran wax, Montan-ester waxes, Sasol waxes, jojoba esters, and the like.

Other wax components can be certain fats (including mono-, di- and triglycerides and fatty acid alkylesters), fatty alcohols, fatty acids, including substituted fatty acids (in particular hydroxy substituted fatty acids, for example, 12-hydroxystearic acid), dialkyl(ene)ethers, dialkyl(ene) carbonates, dicarboxylic acids (in particular the C₁₆-C₄₀-dialkylesters of dicarboxylic acids, e.g. the C₁₆-C₄₀-alkyl stearates, C₁₈-C₃₈-alkylhydroxystearyl stearates or C₂₀-C₄₀-alkyl erucates) and hydroxy fatty alcohols. Still further wax components are selected from the group of aromatic carbonic acids, tricarboxylic acids, or from the group of lactides of long-chained hydroxycarbonic acids. Myristyl lactate is a suitable carrier. Further wax components that can be used are C₃₀-C₅₀ alkyl bees wax; tri-C₁₆-C₄₀-alkyl citrates, e.g. tristearyl citrate, triisostearyl citrate, trilauryl citrate; ethyleneglycol di fatty acid esters, in particular the ethylene glycol di-C₁₂-C₃₀-fatty acid esters, e.g. ethylene glycol dipalmitate, ethyleneglycol distearate, and ethyleneglycol di(12-hydroxystearate).

Other suitable carriers include materials that act as solidifying agents, including some of the materials described hereinbefore. Suitable solidifying agent(s) in the lotion compositions of the present invention can function to help solidify the composition so that the composition is a solid at room temperature and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents can be selected from alkyl siloxanes, polymers, hydrogenated vegetable oils having a melting point of 35° C. or greater, fatty acid esters with a melting point of 35° C. or greater, alkyl hydroxystearates, branched esters, alkoxylated alcohols and alkoxylated carboxylic acid. Additionally, the solidifying agents can be selected from animal, vegetable and mineral waxes and alkyl silicones. Examples of suitable solidifying agents include, but are not limited to, the following: alkyl silicones, alkyl trimethylsilanes, beeswax, behenyl behenate, behenyl benzoate, C24-C28 alkyl dimethicone, C30 alkyl dimethicone, cetyl methicone, stearyl methicone, cetyl dimethicone, stearyl dimethicone, cerotyl dimethicone, candelilla wax, carnuba, synthetic carnuba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C20-C40 alkyl behenate, C2-C5 lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di C2-C5 alkyl fumerate, dibehenyl fumerate, myristyl lactate, myristyl lignocerate, myristyl myristate, myristyl stearate, lauryl stearate, octyidodecyl stearate; octyidodecyl stearoyl stearate, oleyl arachidate, oleyl stearate, tridecyl behenate, tridecyl stearate, tridecyl stearoyl stearate, pentaerythrityl tetrabehenate, penteerythritylhydrogenated rosinate, pentaerythrityl distearate, pentaerythrityltetraabeite, penteerythrityl tetracocoate, penteerythrityl tetraperlargonate, pentserythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C30-C60 fatty alcohols, C20+ fatty amides, polypropylene, polystyrene, polybutane, polybutylene terephthalate, polydipentane, polypropylene, zinc stearate, dodecyl laurate, stearyl palmitate, octadecyl hexedecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C20-C40 alkyl hydroxystearyl stearate, and mixtures of such compounds.

In preparing a lotioned catamenial device according to the present invention, the lotion composition is applied to the outer surface of the topsheet. Any of a variety of application methods that distribute lubricious materials having a molten or liquid consistency can be used. Suitable methods include but are not limited to spraying, printing (e.g., flexographic printing), coating (e.g., gravure coating), extrusion, dipping, or combinations of these application techniques, e.g. spraying the lotion composition on a rotating surface, such as a calender roll, that then transfers the composition to the outer surface of the sanitary napkin topsheet. Additionally, the manner of applying the lotion composition to a portion of a catamenial device can be such that the substrate or component does not become saturated with the lotion composition.

The minimum level of the lotion composition to be applied to a component of the catamenial device is preferably an amount effective for reducing the adherence of menses to that component.

The lotion composition can also be applied nonuniformly to a component or layer of the catamnial device. By “nonuniform” is meant that the amount, pattern of distribution, etc. of the lotion composition can vary. For example, a non-uniform pattern of lotion composition 22 is shown in FIG. 3. A non-uniform application of lotion composition may be a random pattern of regular shapes such as dots, stripes, rectangles, or other irregular pattern.

The lotion composition can be applied to the catamenial device at any point during assembly. For example, the lotion composition can also be applied to the outer surface of the topsheet before it is combined with the other raw materials to form a finished catamenial device.

Lotion compositions of the present invention can be applied by printing methods, or continuous spray or extrusion as is known in the art, or as is disclosed in U.S. Pat. No. 5,968,025.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a liquid pervious topsheet, said topsheet having an inner surface oriented toward the interior of said absorbent article and an outer surface oriented toward the skin of the wearer when said absorbent article is being worn,
a backsheet joined to said topsheet, said backsheet having an inner surface oriented toward the interior of said absorbent article and an outer surface oriented toward the garment of the wearer when said absorbent article is being worn;
an absorbent core disposed between said topsheet and said backsheet, said absorbent core having an inner surface oriented toward the skin of the wearer when said absorbent article is being worn and an outer surface oriented toward the garment of the wearer when said absorbent article is being worn;
and a lotion composition applied to at least a portion of said outer surface of said topsheet, wherein said lotion composition comprises a polypropylene glycol material selected from the group consisting of polypropylene glycol copolymer, polypropylene glycol surfactant, and mixtures thereof.

2. The absorbent article of claim 1, wherein said polypropylene glycol material is polypropylene glycol copolymer, and wherein said polypropylene glycol copolymer comprises an internal block component and a terminal block component, wherein said internal block component has a formula: 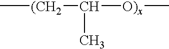
and said terminal block component has a formula: 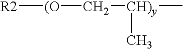
wherein x is from 2 to 120, y is from 2 to 100, and R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate.

3. The absorbent article of claim 1, wherein said polypropylene glycol material is polypropylene glycol copolymer, and wherein said polypropylene glycol copolymer is selected from the group consisting of PPG-12 dimethicone, bis-PPG-15 dimethicone/IPDI copolymer, PPG/polycaprolactone block copolymer, PPG/polybutanediol/PEG triblock copolymer, polyethylimine/PPG copolymer; polyacrylic acid-g-PPG graft copolymer, and mixtures thereof.

4. The absorbent article of claim 1, wherein said polypropylene glycol material is polypropylene glycol surfactant, and wherein said polypropylene glycol surfactant has a formula: 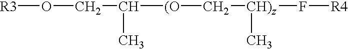
wherein R3 is hydrogen, alkyl, alkyl carbonyl, alkylenelamine, alkylenelamide, alkylene phosphate, alkylene carboxylic acid, alkylene sulfonate salt or alkylene quat with a maximum number of carbon elements of less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100; and F is a functional group selected from the group consisting of an ether group, an ester group, an amine group, an amide group, and a phosphate ester group.

5. The absorbent article of claim 1, wherein said polypropylene glycol material is polypropylene glycol surfactant, and wherein said polypropylene glycol surfactant has a formula: 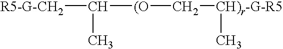
wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120; and G is an ether, an ester, an amine, or an amide linkage.

6. The absorbent article of claim 1, wherein said polypropylene glycol material is polypropylene glycol surfactant, and wherein said polypropylene glycol surfactant is selected from the group consisting of PPG-30 cetyl ether, PPG-20 methyl glucose ether distearate, PPG-20 methyl glucose ether acetate, PPG-20 sorbitan tristearate, PPG-20 methyl glucose ether distearate, PPG-20 distearate, PPG-15 stearyl ether, PPG-15 stearyl ether benzoate, PPG-15 isohexadecyl ether, PPG-15 stearate, PPG-15 dicocoate, PPG-12 dilaurate, PPG-11 stearyl ether, PPG-10 cetyl ether, PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether, PPG-3 myristyl ether propionate, PPG-3 benzyl ether myristate, PPG-3 hydrogenated castor oil, PPG-3-hydroxyethyl soyamide, PPG-2 cocamide, PPG-2 lanolin alcohol ether, PPG-1 coconut fatty acid isopropanolamide, and mixtures thereof.

7. The absorbent article of claim 6, wherein said polypropylene glycol surfactant is PPG-15 stearyl ether.

8. The absorbent article of claim 1, wherein said absorbent article is a catamenial device selected from the group consisting of a sanitary napkin and a pantiliner.

9. The absorbent article of claim 1, wherein said topsheet is selected from the group consisting of a nonwoven material and a formed film material.

10. The absorbent article of claim 1, wherein said topsheet is hydrophilic.

11. The absorbent article of claim 1, wherein said topsheet is hydrophobic.

12. The absorbent article of claim 1, wherein said lotion composition is applied in an amount of from about 0.01 grams per square meter to about 100 grams per square meter of area covered by the lotion composition.

13. An absorbent article comprising:
a liquid pervious topsheet, said topsheet having an inner surface oriented toward the interior of said absorbent article and an outer surface oriented toward the skin of the wearer when said absorbent article is being worn,
a backsheet joined to said topsheet, said backsheet having an inner surface oriented toward the interior of said absorbent article and an outer surface oriented toward the garment of the wearer when said absorbent article is being worn;
an absorbent core disposed between said topsheet and said backsheet, said absorbent core having an inner surface oriented toward the skin of the wearer when said absorbent article is being worn and an outer surface oriented toward the garment of the wearer when said absorbent article is being worn;
and a lotion composition applied to at least a portion of said outer surface of said topsheet, wherein said lotion composition consists essentially of a polypropylene glycol material and optionally a carrier.

14. The absorbent article of claim 13, wherein said polypropylene glycol material is polypropylene glycol homopolymer, and wherein said polypropylene glycol homopolymer has a formula: 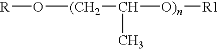
wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and n is from 3 to 160.

15. The absorbent article of claim 13, wherein said polypropylene glycol material is polypropylene glycol.

16. The absorbent article of claim 13, wherein said polypropylene glycol has a number average molecular weight of from about 400 to about 10,000.

17. The absorbent article of claim 13, wherein said absorbent article is a catamenial device selected from the group consisting of a sanitary napkin and a pantiliner.

18. The absorbent article of claim 13, wherein said topsheet is selected from the group consisting of a nonwoven material and a formed film material.

19. The absorbent article of claim 13, wherein said topsheet is hydrophilic.

20. The absorbent article of claim 13, wherein said topsheet is hydrophobic.

21. The absorbent article of claim 13, wherein said lotion composition is applied in an amount of from about 0.01 grams per square meter to about 100 grams per square meter of area covered by the lotion composition.

22. The absorbent article of claim 13, wherein said lotion composition consists essentially of said polypropylene glycol material and said carrier.

23. The absorbent article of claim 13, wherein said lotion composition consists essentially of said polypropylene glycol material.
